(19) United States
(12) Patent Application Publication
(10) Pub. No.: US 2004/0138511 A1
(43) Pub. Date: Jul. 15, 2004

(54) Aromatic alkylation process with direct recycle

(76) Inventors: James Butler, Deer Park, TX (US); James Merrill, Deer Park, TX (US); Kevin Kelly, Friendswood, TX (US)

Correspondence Address: David J. Alexander, Fina Technology, Inc., P.O. Box 674411, Houston, TX, 77267-4411, US

(21) Appl. No.: 10/340,082

(22) Filed: Jan. 10, 2003

(51) Int. Cl. C07C 5/22; C07C 15/12

(52) U.S. Cl. 585/323; 585/467; 585/475

(57) ABSTRACT

Process for the alkylation of an aromatic substrate with partial recycling of the alkylated product. A feedstock comprising an aromatic substrate and an alkylating agent is introduced into an alkylation reaction zone and into contact with a molecular sieve catalyst to produce an alkylation product which is withdrawn from the alkylation reaction zone and split into two portions. A first portion is recycled back to the alkylation reaction zone and supplied to the alkylation zone. A second portion is supplied to a suitable recovery zone for the separation of alkylated aromatic components from the unreacted aromatic substrate. The alkylation reaction zone may be operated under conditions in which the aromatic substrate is in the supercritical phase, and may comprise a plurality of catalyst beds wherein the recycled portion of the alkylation reaction product is subdivided into subproducts with one subproduct recycled to the inlet of the alkylation reaction zone and another subproduct introduced into the alkylation reaction zone between catalyst beds.

### FIELD OF THE INVENTION

This invention relates to the alkylation in a reactor of an aromatic substrate and more particularly to the ethylation of benzene with recycle of a portion of the product to the reactor which is operated under the conditions in which the benzene is in the liquid or supercritical phase.

### BACKGROUND OF THE INVENTION

The alkylation of an aromatic substrate such as benzene or an alkyl benzene such as to produce an alkyl benzene or polyalkyl benzene is well known in the art. For example, the alkylation of benzene with ethylene over a molecular sieve catalyst is a well-known procedure for the production of ethylbenzene. Typically, the alkylation reaction is carried out in a multistage reactor involving the interstage injection of ethylene and benzene to produce an output from the reactor that involves a mixture of monoalkyl and polyalkylbenzene. The principal monoalkylbenzene is, of course, the desired ethylbenzene product. Polyalkylbenzenes include diethylbenzene, triethylbenzene, and xylenes.

In many cases, it is desirable to operate the alkylation reactor in conjunction with the operation of a transalkylation reactor in order to produce additional ethylbenzene through the transalkylation reaction of polyethylbenzene with benzene. The alkylation reactor can be connected to the transalkylation reactor in a flow scheme involving one or more intermediate separation stages for the recovery of ethylene, ethylbenzene, and polyethylbenzene.

Transalkylation may also occur in the initial alkylation reactor. In this respect, the injection of ethylene and benzene between stages in the alkylation reactor not only results in additional ethylbenzene production but also promotes transalkylation within the alkylation reactor in which benzene and diethylbenzene react through a disproportionation reaction to produce ethylbenzene.

Various phase conditions may be employed in the alkylation and transalkylation reactors. Typically, the transalkylation reactor will be operated under liquid phase conditions, i.e., conditions in which the benzene and polyethylbenzene are in the liquid phase, and the alkylation reactor is operated under gas phase conditions, i.e., pressure and temperature conditions in which the benzene is in the gas phase. However, liquid phase conditions can be used where it is desired to minimize the yield of undesirable by-products from the alkylation reactor.

### SUMMARY OF THE INVENTION

In accordance with the present invention there is provided a process for the alkylation of an aromatic substrate with partial recycling of the alkylated product. In carrying out the invention there is provided an alkylation reaction zone containing a molecular sieve aromatic alkylation catalyst. A feedstock comprising an aromatic substrate and an alkylating agent is introduced into the alkylation reaction zone and into contact with the catalyst therein. The alkylation zone is operated under temperature and pressure conditions effective to cause alkylation of the aromatic substrate in the presence of the molecular sieve catalyst to produce an alkylation product which is withdrawn from the alkylation reaction zone. The alkylation product typically will comprise a mixture of the aromatic substrate and monoakylated and polyalkylated aromatic components. The product withdrawn from the alkylation reaction zone is split into two portions. A first portion of the alkylation product is recycled back to the alkylation reaction zone and supplied to the alkylation zone along with the aromatic substrate and the alkylating agent. A second portion of the alkylation product is supplied to a suitable recovery zone where the separation of alkylated aromatic components from the unreacted aromatic substrate is accomplished.

In the normal course of operation a substantial portion of the alkylated product is recycled back to the alkylation reaction zone. Preferably, the weight ratio of the first portion which is recycled and the second portion which is supplied to the recovery zone is at least 1:1 and more preferably at least 2:1. Normally, the upper limit of the weight ratio of the first portion to the second portion will be about 5:1 with an upper limit of 10:1 being preferred.

In a preferred embodiment of the invention the alkylation reaction zone is operated to provide the aromatic substrate to be in the liquid phase or the supercritical phase. In a specifically preferred embodiment, the aromatic substrate is in the supercritical phase.

In a particular aspect of the invention the aromatic substrate is benzene, and the alkylating agent is ethylene, with the molecular sieve catalyst in the alkylation reaction zone comprising zeolite beta. Preferably, the zeolite beta alkylation catalyst is a rare earth modified zeolite beta, more specifically a lanthanum modified zeolite beta or a cerium modified zeolite beta.

The alkylation reaction zone may comprise a single catalyst bed or a plurality of catalyst beds. Preferably, at least a predominant portion of the alkylation catalyst is contained within a single catalyst bed in the alkylation reaction zone. Where a plurality of catalyst beds are employed, the recycled portion of the alkylation reaction product is subdivided into subproducts with one subproduct recycled to the inlet of the alkylation reaction zone and another subproduct introduced into the alkylation reaction zone between catalyst beds.

In a further aspect to the invention a recycle procedure as described above is employed in an integrated process comprising an alkylation reaction zone and a transalkylation zone. In a specific embodiment of the invention a feedstock comprising benzene and a C₂-C₄ alkylating agent is supplied to the alkylation reaction zone which is operated under liquid phase or supercritical phase conditions to produce an alkylation product containing a mixture of benzene and monoalkyl and polyalkyl benzenes. A first portion of the alkylation product recovered from the alkylation reaction zone is recycled to the alkylation reaction zone as described previously. The second portion is supplied to an intermediate recovery zone for the recovery of alkyl benzene and the recovery of a polyalkylated aromatic component including a dialkyl benzene. At least a portion of the polyalkylated aromatic component is supplied to a transalkylation reaction zone containing a molecular sieve transalkylation catalyst along with benzene. Preferably, the transalkylation reaction zone is operated under conditions to cause disproportionation of the polyalkylated aromatic to produce a disproportionation product having a reduced dialkyl benzene content and an enhanced alkyl benzene content. Preferably, the benzene recovered from the alkylation product in the separation and recovery zone is recycled to the alkylation reaction zone.

### BRIEF DESCRIPTION OF THE DRAWINGS

FIG. 1 is an idealized schematic block diagram of an alkylation/transalkylation process embodying the present invention.

FIG. 2 is a schematic illustration of a preferred embodiment of the invention incorporating separate parallel-connected alkylation and transalkylation reactors with an intermediate multi-stage recovery zone for the separation and recycling of components.

FIG. 3 is a schematic illustration of an alkylation reactor comprising a single catalyst bed with recycle of a portion of the reactor output.

FIG. 4 is a schematic illustration of a modified form of an alkylation reactor employing two catalyst beds with a portion of the recycled product being directed between the catalyst beds.

FIG. 5 is a graph illustrating the benzene rate and the benzene/ethylene molar ratio of a feedstock applied to an alkylation reactor.

FIG. 6 is a graph illustrating the percent of bed used in the experimental work.

FIG. 7 is a graph illustrating the ethyl benzene yield versus time for the reactor.

FIG. 8 is a graph illustrating the ethyl benzene yield and the diethyl benzene yield over time in the product from the alkylation reactor.

FIG. 9 is a graph of the propyl benzene yield and the butyl benzene yield over time in the product from the alkylation reactor.

FIG. 10 is a graph illustrating the triethyl benzene yield versus time in the product from the alkylation reactor.

FIG. 11 is a graph showing the heavy byproduct yield from the reactor plotted as a function of time.

### DETAILED DESCRIPTION OF THE INVENTION

The present invention involves the alkylation of an aromatic substrate such as benzene over a molecular sieve alkylation catalyst in an alkylation reaction and with recycle of a portion of the product from the alkylation reactor directly back to the alkylation reactor. The alkylation reactor is operated under conditions to control and desirably minimize the yield of by-products in the alkylation reaction zone. The feedstock supplied to the alkylation reaction zone comprises benzene as a major component and ethylene as a minor component. Typically, the benzene and ethylene streams will be combined to provide a benzene-ethylene mixture into the reaction zone. The benzene stream, which is mixed with the ethylene either before or after introduction into the reaction zone, should be a relatively pure stream containing only very small amounts of contaminants. The benzene stream should contain at least 95 wt. % benzene. Preferably, the benzene stream will be at least 98 wt. % benzene with only trace amounts of such materials as toluene, ethyl benzene, and C₇ aliphatic compounds that cannot readily be separated from benzene. The alkylation zone may be operated under gas phase conditions but preferably is under liquid phase or supercritical phase conditions. Preferably, the alkylation reaction zone is operated under supercritical conditions, that is, pressure and temperature conditions which are above the critical pressure and critical temperature of benzene. Specifically, the temperature in the alkylation zone is at or above 310° C., and the pressure is at or above 550, psia preferably at least 600 psia. Preferably, the temperature in the alkylation reactor will be maintained at an average value within the range of 320-350° C. and a pressure within the range of 550-1600 psia and more preferably 600-800 psia. The critical phase alkylation reaction is exothermic with a positive temperature gradient from the inlet to the outlet of the reactor, typically providing a temperature increment increase within the range of about 20-100° C.

The operation of the alkylation reaction zone in the supercritical region enables the alkylation zone to be operated under conditions in which the benzene-ethylene mole ratio can be maintained at relatively low levels, usually somewhat lower than the benzene-ethylene mole ratio encountered when the alkylation reaction zone is operated under liquid phase conditions. In most cases, the benzene-ethylene mole ratio will be within the range of 1-15. Preferably, the benzene/ethylene mole ratio will be maintained during at least part of a cycle of operation at a level within the lower end of this range, specifically, at a benzene-ethylene mole ratio of less than 10. Thus, operation in the supercritical phase offers the advantages of gas phase alkylation in which the benzene-ethylene ratio can be kept low but without the problems associated with by-product formation, specifically xylene formation, often encountered in gas-phase alkylation. At the same time, operation in the super critical phase offers the advantages accruing to liquid phase alkylation in which the by-product yield is controlled to low levels. The pressures required for operation in the super critical phase are not substantially greater than those required in liquid phase alkylation, and the benzene in the supercritical phase functions as a solvent to keep the molecular sieve catalyst clean and to retard coking leading to deactivation of the catalyst.

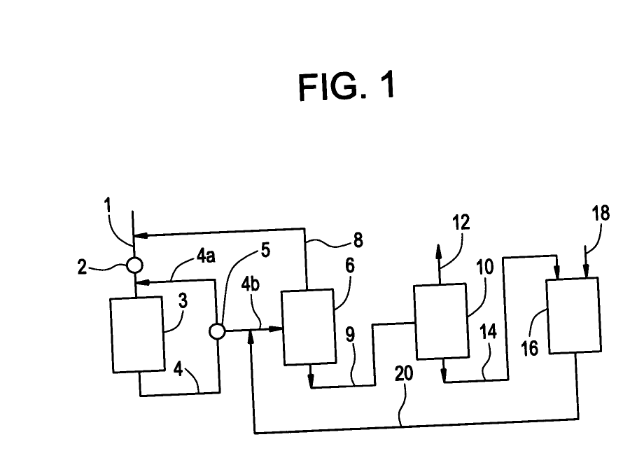

Turning now to FIG. 1, there is illustrated a schematic block diagram of an alkylation/transalkylation process employing the present invention. As shown in FIG. 1, a product stream comprising a mixture of ethylene and benzene in a mole ratio of benzene to ethylene about 1 to 15 is supplied via line 1 through a heat exchanger 2 to an alkylation reaction zone 3 which may single stage or multistage. Alkylation zone 3 preferably comprises parallel reactors which contain a molecular sieve alkylation catalyst as described herein. The alkylation zone 3 can be vapor phase or liquid phase but preferably is operated at temperature and pressure conditions to maintain the alkylation reaction in the supercritical phase, i.e. the benzene is in the supercritical state, and at a feed rate to provide a space velocity enhancing diethylbenzene production while retarding by-products production. Preferably, the space velocity of the benzene feed stream will be within the range of 10-150 hrs⁻¹ LHSV per catalyst bed, and more specifically 40-100 hrs⁻¹ LHSV per catalyst bed.

The output from the alkylation reactor 3 is supplied via line 4 to a splitter valve 5 where the alkylation product is separated into two portions. A first portion of the alkylation product is recycled back to the alkylation reactor via line 4a. A second portion of the alkylation product is supplied via line 4b to an intermediate benzene separation zone 6 that may take the form of one or more distillation columns. Benzene is recovered through line 8 and recycled through line 1 to the alkylation reactor. The bottoms fraction from the benzene separation zone 6, which includes ethylbenzene and polyalkylated benzenes including polyethylbenzene is supplied via line 9 to an ethylbenzene separation zone 10. The ethylbenzene separation zone may likewise comprise one or more sequentially connected distillation columns. The ethylbenzene is recovered through line 12 and applied for any suitable purpose, such as in the production of vinyl benzene. The bottoms fraction from the ethylbenzene separation zone 10, which comprises polyethylbenzene, principally diethylbenzene, is supplied via line 14 to a transalkylation reactor 16. Benzene is supplied to the transalkylation reaction zone through line 18. The transalkylation reactor, which preferably is operated under liquid phase conditions, contains a molecular sieve catalyst, preferably zeolite-Y, which typically has a somewhat larger pore size than the molecular sieve used in the alkylation reaction zone. The output from the transalkylation reaction zone is recycled via line 20 to the benzene separation zone 6.

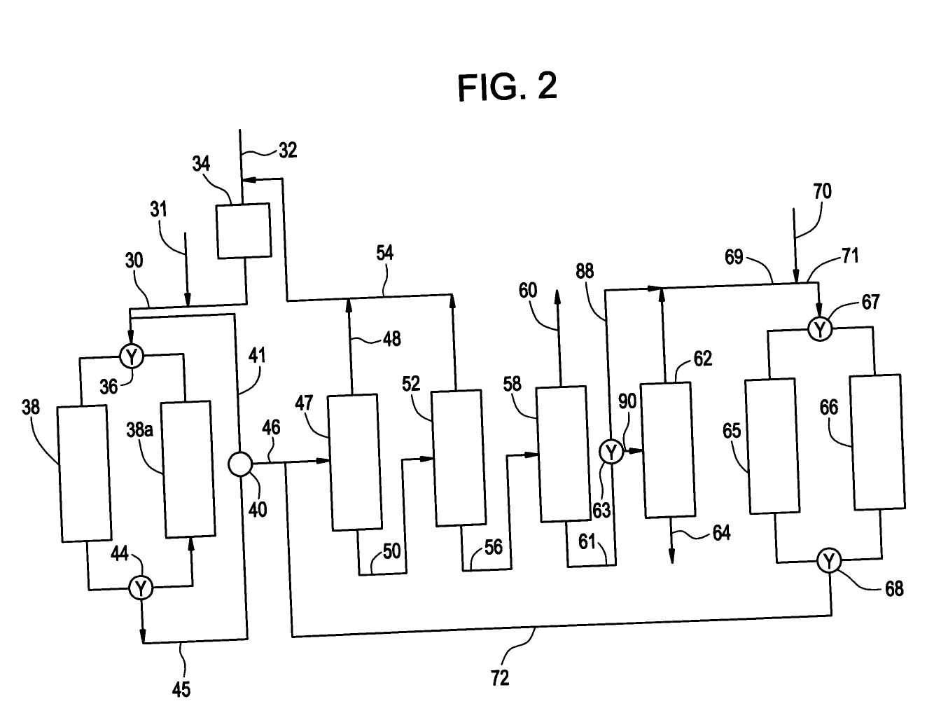

Referring now to FIG. 2, there is illustrated in greater detail a suitable system incorporating a multi-stage intermediate recovery zone for the separation and recycling of components involved in the alkylation and transalkylation process. As shown in FIG. 2, an input feed stream is supplied by fresh ethylene through line 31 and fresh benzene through line 32. The fresh benzene stream, supplied via line 32, is of high purity containing at least 98 wt. %, preferably about 99 wt. % benzene with no more than 1 wt. % other components. Typically, the fresh benzene stream will contain about 99.5 wt. % benzene, less than 0.5% ethylbenzene, with only trace amounts of non-aromatics and toluene. Line 32 is provided with a preheater 34 to heat the benzene stream consisting of fresh and recycled benzene to the desired temperature for the alkylation reaction. The feed stream is supplied through a two-way, three-position valve 36 and inlet line 30 to the top of one or both parallel liquid phase or critical phase alkylation reactors 38 and 38A each of which contains the desired molecular sieve alkylation catalyst. For super critical phase operation, the reactors are operated at a temperature, preferably within the range of 310°-350° C. inlet temperature and at pressure conditions of about 550 to 1000 psia, to maintain the benzene in the critical phase. For liquid phase the temperature will normally be within the range of 150-300° C. and the pressure within the range of 450-1000 psia.

In normal operation of the system depicted in FIG. 2, both reaction zones 38 and 38A may, during most of a cycle of operation, be operated in a parallel mode of operation in which they are both in service at the same time. In this case, valve 36 is configured so that the input stream in line 30 is roughly split in two to provide flow to both reactors in approximately equal amounts. Periodically, one reactor can be taken off-stream for regeneration of the catalyst. Valve 36 is then configured so that all of the feed stream from line 30 can be supplied to reactor 38 while the catalyst in reactor 38A is regenerated and visa versa. The regeneration procedure will be described in detail below but normally will take place over a relatively short period of time relative to the operation of the reactor in parallel alkylation mode. When regeneration of the catalyst in reactor 38A is completed, this catalyst can then be returned on-stream, and at an appropriate point, the reactor 38 can be taken off-stream for regeneration. This mode of operation involves operation of the individual reactors at relatively lower space velocities for prolonged periods of time with periodic relatively short periods of operation at enhanced, relatively higher space velocities when one reactor is taken off-stream. By way of example, during normal operation of the system with both reactors 38 and 38A on-stream, the feed stream is supplied to each reactor to provide a space velocity of about 10-45 hrs.⁻¹ LHSV. When reactor 38A is taken off-stream and the feed rate continues unabated, the space velocity for reactor 38 will approximately double to 50-90 hr.⁻¹ LHSV. When the regeneration of reactor 38A is completed, it is placed back on-stream, and again the feed stream rate space velocity for each reactor will decrease to 10-45 hr.⁻¹ until such point as reactor 38 is taken off-stream, in which case the flow rate to reactor 38A will, of course, increase, resulting again in a transient space velocity in reactor 38 of about 50-90 hr⁻¹ LHSV.

The effluent stream from one or both of the alkylation reactors 38 and 38A is supplied through a two-way, three-position outlet valve 44 and outlet line 45 to a splitter valve 40 which is analogous to valve 5 shown in FIG. 1. A first portion of the alkylated product is recycled via line 41 to one or both alkylation reactors 38 and 38a, as described in greater detail hereinafter. A second portion of the alkylation product is supplied via line 46 to a two-stage benzene recovery zone which comprises as the first stage a prefractionation column 47. Column 47 is operated to provide a light overhead fraction including benzene which is supplied via line 48 to the input side of heater 34 where it is mixed with benzene in line 32 and then to the alkylation reactor input line 30. A heavier liquid fraction containing benzene, ethylbenzene and polyethylbenzene is supplied via line 50 to the second stage 52 of the benzene separation zone. Stages 47 and 52 may take the form of distillation columns of any suitable type, typically, columns having from about 20-60 stages. The overhead fraction from column 52 contains the remaining benzene, which is recycled via line 54 to the alkylation reactor input. Thus, lines 48 and 54 correspond to the output line 8 of FIG. 1. The heavier bottoms fraction from column 52 is supplied via line 56 to a secondary separation zone 58 for the recovery of ethylbenzene. The overhead fraction from column 58 comprises relatively pure ethylbenzene, which is supplied to storage or to any suitable product destination by way of line 60. By way of example, the ethylbenzene may be used as a feed stream to a styrene plant in which styrene is produced by the dehydrogenation of ethylbenzene. The bottoms fraction containing polyethylbenzenes, heavier aromatics such as cumene and butylbenzene, and normally only a small amount of ethylbenzene is supplied through line 61 to a tertiary polyethylbenzene separation zone 62. As described below, line 61 is provided with a proportioning valve 63 which can be used to divert a portion of the bottoms fraction directly to the transalkylation reactor. The bottoms fraction of column 62 comprises a residue, which can be withdrawn from the process via line 64 for further use in any suitable manner. The overhead fraction from column 62 comprises a polyalkylated aromatic component containing diethylbenzene and a smaller amount of triethylbenzene and a minor amount of ethylbenzene is supplied to an on stream transalkylation reaction zone. Similarly as described above with respect to the alkylation reactors, parallel transalkylation reactors 65 and 66 are provided through inlet and outlet manifolding involving valves 67 and 68. Both of reactors 65 and 66 can be placed on stream at the same time so that both are in service in a parallel mode of operation. Alternatively, only one transalkylation reactor can be on-stream with the other undergoing regeneration operation in order to burn coke off the catalyst beds. By minimizing the amount of ethylbenzene recovered from the bottom of column 58, the ethylbenzene content of the transalkylation feed stream can be kept small in order to drive the transalkylation reaction in the direction of ethylbenzene production. The polyethylbenzene fraction withdrawn overhead from column 62 is supplied through line 69 and mixed with benzene supplied via line 70. This mixture is then supplied to the on-line transalkylation reactor 65 via line 71. Preferably, the benzene feed supplied via line 70 is of relatively low water content, about 0.05 wt. % or less. Preferably, the water content is reduced to a level of about 0.02 wt. % or less and more preferably to less than 0.01 wt. % or less. The transalkylation reactor is operated as described before in order to maintain the benzene and alkylated benzenes within the transalkylation reactor in the liquid phase. Typically, the transalkylation reactor may be operated to provide an average temperature within the transalkylation reactor of about 65°-290° C. and an average pressure of about 600 psi. The preferred catalyst employed in the transalkylation reactor is zeolite Y. The weight ratio of benzene to polyethylbenzene should be at least 1:1 and preferably is within the range of 1:1 to 4:1.

The output from the transalkylation reactor or reactors containing benzene, ethylbenzene, and diminished amounts of polyethylbenzene is recovered through line 72. In one embodiment of the invention, line 72 will be connected to the inlet lines 46 for recycle to the prefractionation column 47 as shown. However, the effluent from the liquid-phase transalkylation reactor may be supplied to either or both of distillation columns 47 and 52.

Another embodiment of the invention involves applying the output from the transalkylation reactor directly back to the input to the alkylation reactor. Thus, all or part of the transalkylation effluent may be recycled back to line 41 shown FIG. 2. Alternatively, all of the transalkylation reactor output may be applied to line 41 or a portion may be applied to line 41, and the other applied through a splitter valve to line 46. This embodiment of the invention is illustrated in FIG. 2A, which shows the flow diagram of FIG. 2 with modifications in the outlet line 72 from the transalkylation reactor. As indicated, line 72 is supplied to a two-way, two-position valve 72(a). The output from valve 72(a) may be applied in its entirety through line 72(b) to line 41, and ultimately into the alkylation reactors 38, 38(a). Alternatively, the output for valve 72(b) may be split in whatever proportions are desired with a portion applied via line 72b to line 41 and another portion applied via line 72c to line 46.

Returning to the operation of the separation system, in one mode of operation the entire bottoms fraction from the ethylbenzene separation column 58 is applied to the tertiary separation column 62 with overhead fractions from this zone then applied to the transalkylation reactor. This mode of operation offers the advantage of relatively long cycle lengths of the catalyst in the transalkylation reactor between regeneration of the catalyst to increase the catalyst activity. Another mode of operation of the invention achieves this advantage by supplying a portion of the output from the ethylbenzene separation column 58 through valve 63 directly to the transalkylation reactor.

As shown in FIG. 2, a portion of the bottoms fraction from the secondary separation zone 58 bypasses column 62 and is supplied directly to the transalkylation reactor 65 via valve 63 and line 88. A second portion of the bottoms fraction from the ethylbenzene column is applied to the tertiary separation column 62 through valve 63 and line 90. The overhead fraction from column 62 is commingled with the bypass effluent in line 88 and the resulting mixture is fed to the transalkylation reactor via line 67. In this mode of operation a substantial amount of the bottoms product from column 58 can be sent directly to the transalkylation reactor, bypassing the polyethylbenzene column 62. Normally, the weight ratio of the first portion supplied via line 88 directly to the transalkylation reactor to the second portion supplied initially via line 90 to the polyethylbenzene would be within the range of about 1:2 to about 2:1. However, the relative amounts may vary more widely to be within the range of a weight ratio of the first portion to the second portion in a ratio of about 1:3 to 3:1.

The alkylation reactor or reactors employed in the present venture can be multistage reactors of the type commonly employed in benzene alkylation processes or they may take the form of a single stage reactor or a reactor having a plurality but still a limited number of catalyst beds. In a preferred embodiment of the invention, the alkylation reactor will be configured so that the alkylation catalyst resides in a single catalyst bed within the reactor or configured in a manner in which a predominant portion of the alkylation catalyst resides within a single catalyst bed within the reactor. The operation of the invention in conjunction with a single catalyst bed or a limited number of catalyst bed functions to keep the reaction in the liquid phase or supercritical phase by controlling the exotherm of the reaction similarly as accomplished by the interstage injection of ethylene as a quench fluid between catalyst stages.

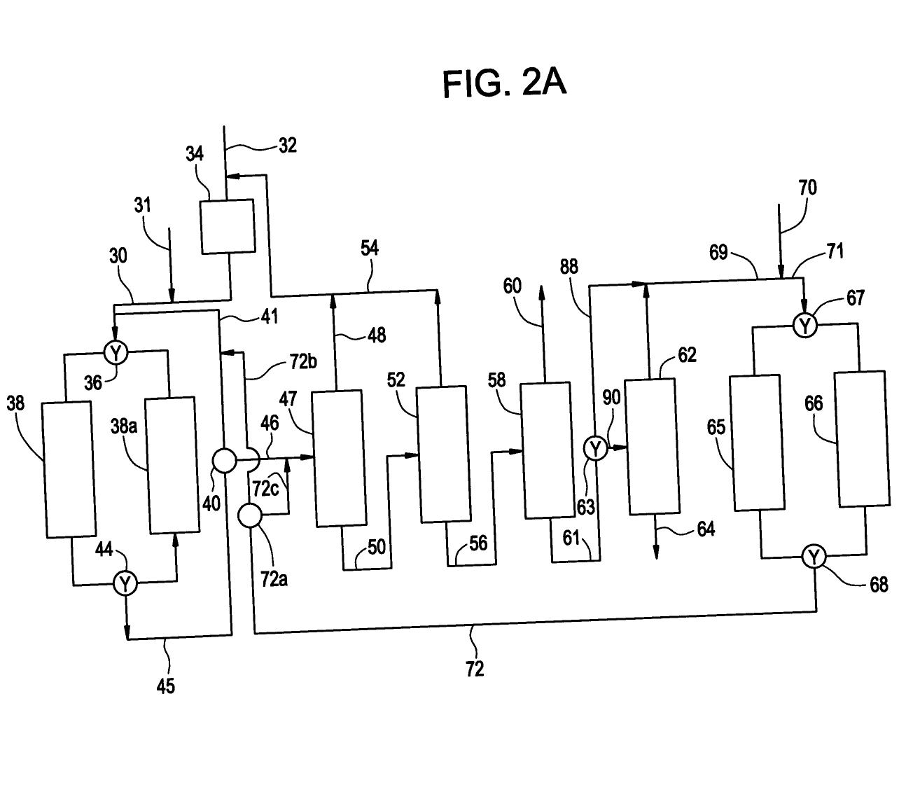

Turning now to FIG. 3 there is illustrated a single stage reactor configuration suitable for use in the present invention. As shown in FIG. 3, reactor 91 is a single stage reactor having a catalyst bed 92 supported within the reactor to provide an inlet plenum 93 and an outlet plenum 94. A portion of the product recovered from the bottom of the reactor is recycled to an inlet line 95 via recycle line 96 and introduced into the reactor at the inlet plenum 93. Additional ethylene and benzene is supplied to the inlet of the reactor via line 96.

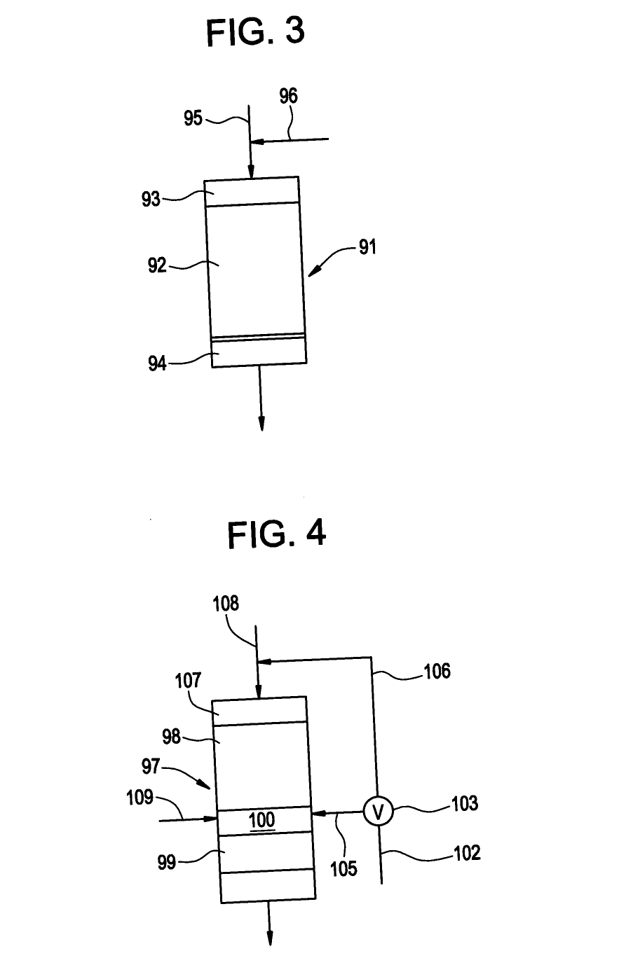

FIG. 4 is a schematic illustration of a multi-stage reactor 97 having an initial catalyst bed 98, a lower catalyst bed 99, with an interior plenum chamber 100 interposed between the upper and lower catalyst beds. In FIG. 4, the recycled portion of the alkylation product recovered from the bottom of reactor 97 is applied via line 102 to a splitter valve 103 where it is divided into two subportions. One subportion is applied via line 105 to the intermediate plenum 100 and the other subportion of the product is supplied via line 106 to the inlet plenum 107 of the reactor. The fresh feedstock comprising a mixture of benzene and ethylene is supplied via line 108 to the reactor inlet plenum 107, and also supplied via line 109 to the intermediate plenum 100.

In the embodiment illustrated in FIG. 4, the reactor bed 98 contains substantially more catalysts than the lower reactor bed 99, and in this case the recycle stream applied via line 106 will be proportionately greater than the portion of the recycle stream applied via line 105. However, the volume of catalysts in beds 98 and 99 may be approximately equal in which case the subportions circulated to the reactor via lines 105 and 106 will likewise be approximately equal.

Where a multistage reactor is employed, it can involve more than two catalyst beds with interstage injection of the recycle stream between succeeding catalyst beds. The concept of an operation is the same regardless of whether multiple catalyst beds or a single bed reactor is employed. However, the present invention offers a significant advantage in that a single bed alkylation reactor can be employed by virtue of the recycle stream as described previously to obtain results similar to those obtained with multiple stage reactors having a high number of reactor beds.

The molecular sieve catalyst employed in the alkylation reaction zone and the transalkylation reaction zone may be the same or different, but as described below, it usually will be preferred to employ different molecular sieves. The molecular sieve catalyst employed in a liquid phase or critical phase alkylation reactor will normally be of a larger pore size characteristic than catalysts such as silicalite which can be employed in vapor phase alkylation processes. In this regard, the small to intermediate pore size molecular sieves, like silicalite, do not show good alkylation activity in liquid phase or critical phase conditions. Thus, a silicalite molecular sieve of high silica-alumina ratio shows very little activity when employed in the ethylation of benzene under critical phase conditions. However, the same catalyst, when the reactor conditions converted to gas phase conditions in which the benzene in the gas phase shows good alkylation activity.

While a zeolite Y catalyst can be used in the alkylation reactor, preferably, the molecular sieve catalyst employed in the critical phase alkylation reactor is a zeolite beta catalyst, which can be a conventional zeolite beta or a modified zeolite beta of the various types as described below. The zeolite beta catalyst will normally be formulated in extrudate pellets of a size of about ⅛-inch or less, employing a binder such as silica or alumina. A preferred form of binder is silica, which results in catalysts having somewhat enhanced deactivation and regeneration characteristics than zeolite beta formulated with a conventional alumina binder. Typical catalyst formulations may include about 20 wt. % binder and about 80 wt. % molecular sieve.

The catalyst employed in the transalkylation reactor normally will take the form of a zeolite Y catalyst, such as zeolite Y or ultra-stable zeolite Y. As noted above, the zeolite Y type of molecular sieve can also be employed in the critical phase alkylation reactor but normally a zeolite beta type of catalyst is employed.

Various zeolites of the Y and beta types are in themselves well known in the art. For example, zeolite Y is disclosed in U.S. Pat. No. 4,185,040 to Ward, and zeolite beta is disclosed in U.S. Pat. No. 3,308,069 to Wadlinger and U.S. Pat. No. 4,642,226 to Calvert et al.

The zeolite beta employed in the liquid phase or critical phase alkylation reactor can be conventional zeolite beta, or it may be modified zeolite beta of various types described in greater detail below. Preferably, critical phase alkylation is employed with a modified zeolite beta. The zeolite beta employed in the present invention can be a high silica/alumina ratio zeolite beta, a rare earth lanthanide modified beta, specifically cerium or lanthanum-modified zeolite beta, or a ZSM-12 modified zeolite beta as described in detail below.

Basic procedures for the preparation of zeolite beta are well known to those skilled in the art. Such procedures are disclosed in the aforementioned U.S. Pat. No. 3,308,069 to Wadlinger et al and U.S. Pat. No. 4,642,226 to Calvert et al and European Patent Publication No. 159,846 to Reuben, the disclosures of which are incorporated herein by reference. The zeolite beta can be prepared to have a low sodium content, i.e. less than 0.2 wt. % expressed as Na₂O and the sodium content can be further reduced to a value of about 0.02 wt. % by an ion exchange treatment.

As disclosed in the above-referenced U.S. patents to Wadlinger et al., and Calvert et al, zeolite beta can be produced by the hydrothermal digestion of a reaction mixture comprising silica, alumina, sodium or other alkyl metal oxide, and an organic templating agent. Typical digestion conditions include temperatures ranging from slightly below the boiling point of water at atmospheric pressure to about 170° C. at pressures equal to or greater than the vapor pressure of water at the temperature involved. The reaction mixture is subjected to mild agitation for periods ranging from about one day to several months to achieve the desired degree of crystallization to form the zeolite beta. The resulting zeolite beta is normally characterized by a silica to alumina molar ratio (expressed as SiO₂/Al₂O₃) of between about 20 and 50.

The zeolite beta is then subjected to ion exchange with ammonium ions at uncontrolled pH. It is preferred that an aqueous solution of an inorganic ammonium salt, e.g., ammonium nitrate, be employed as the ion-exchange medium. Following the ammonium ion-exchange treatment, the zeolite beta is filtered, washed and dried, and then calcined at a temperature between about 530° C. and 580° C. for a period of two or more hours.

Zeolite beta can be characterized by its crystal structure symmetry and by its x-ray diffraction patterns. Zeolite beta is a molecular sieve of medium pore size, about 5-6 angstroms, and contains 12-ring channel systems. Zeolite beta is of tetragonal symmetry P4₁22, a=12.7, c=26.4 Å (W. M. Meier and D. H. Olson Butterworth, Atlas of Zeolite Structure Types, Heinemann, 1992, p. 58); ZSM-12 is generally characterized by monoclinic symmetry. The pores of zeolite beta are generally circular along the 001 plane with a diameter of about 5.5 angstroms and are elliptical along the 100 plane with diameters of about 6.5 and 7.6 angstroms. Zeolite beta is further described in Higgins et al, “The framework topology of zeolite beta,” Zeolites, 1988, Vol. 8, November, pp. 446-452, the entire disclosure of which is incorporated herein by reference.

The zeolite beta formulation employed in carrying out the present invention may be based upon conventional zeolite beta, such as disclosed in the aforementioned patent to Calvert et al, a lanthamide series-promoted zeolite beta such as a cerium promoted zeolite beta or a lanthanum-modified zeolite beta as disclosed in the aforementioned EP Patent Publication No. 507,761 to Shamshoum et al, or a zeolite beta modified by an intergrowth of ZSM-12 crystals as disclosed in U.S. Pat. No. 5,907,073 to Ghosh. For a further description of procedures for producing zeolite beta useful in accordance with the present invention, reference is made to the aforementioned Patent Nos. 3,308,069 to Wadlinger, 4,642,226 to Calvert, and 5,907,073 to Ghosh and EPA Publication No. 507,761 to Shamshoum, the entire disclosures of which are incorporated herein by reference.

The invention can be carried out with a zeolite beta having a higher silica/alumina ratio than that normally encountered. For example, as disclosed in EPA Publication No. 186,447 to Kennedy, a calcined zeolite beta can be dealuminated by a steaming procedure in order to enhance the silica/alumina ratio of the zeolite. Thus, as disclosed in Kennedy, a calcined zeolite beta having a silica/alumina ratio of 30:1 was subjected to steam treatment at 650° C. and 100% steam for 24 hours at atmospheric pressure. The result was a catalyst having a silica/alumina ratio of about 228:1, which was then subjected to an acid washing process to produce a zeolite beta of 250:1. Various zeolite betas, such as described above, can be subject to extraction procedures in order to extract aluminum from the zeolite beta framework by extraction with nitric acid. Acid washing of the zeolite beta is carried out initially to arrive at a high silica/alumina ratio zeolite beta. This is followed by ion-exchanging lanthanum into the zeolite framework. There should be no subsequent acid washing in order to avoid removing lanthanum from the zeolite.

The same procedure as disclosed in EP 507,761 to Shamshoum, et al for incorporation of lanthanum into zeolite beta can be employed to produce cerium promoted zeolite beta used in the present invention. Thus cerium nitrate may be dissolved in deionized water and then added to a suspension of zeolite beta in deionized water following the protocol disclosed in EP 507,761 for the incorporation of lanthanum into zeolite beta by ion exchange. Following the ion exchange procedure, the cerium exchanged zeolite beta can then be filtered from solution washed with deionized water and then dried at a temperature of 110° C. The powdered cerium exchanged zeolite beta can then be molded with an aluminum or silicon binding agent followed by extrusion into pellet form.

In experimental work carried out respecting the present invention, the reaction of ethylene with benzene under critical phase conditions was carried out employing a single stage alkylation reactor. The reactor operated as a laboratory simulation of the single stage reactor of the type illustrated in FIG. 3. In carrying out the experimental work a cerium promoted zeolite beta having a silica alumina ratio of 150 and a cerium/aluminum atomic ratio of 0.75 was employed. This catalyst was formed employing a silica binder.

The cerium promoted zeolite beta was used in the recycle reactor for a period of about 16 weeks. Throughout the test the inlet temperature of the reactor was about 315° C.±15° C. and the temperature at the outlet of the reactor was about 330° C. 110° C. resulting in an incremental temperature increase across the reactor of about 15-25° C. The reactor was operated at an inlet pressure of about 595-600 PSIG with a pressure gradient across the reactor of only a few pounds per square inch.

The reactor contained 22 grams of the cerium promoted zeolite beta. Benzene was supplied to the top of the reactor at a rate between 3 and 3.5 grams per minute, and ethylene was supplied to provide a benzene ethylene mole ratio within the range of about 3 to 6.5, as described below. The reaction product withdrawn from the reactor was split to provide a recycle ratio of about 5:1 after an initial start-up period. This resulted in an equilibrium condition in which 3 to 3.5 grams per minute of fresh benzene feed was supplied to the reactor, along with about 15 grams per minute of recycled product returned to the front of the reactor. Thus the total output from the reactor was about 18 grams per minute with 3 grams per minute being withdrawn from the process and the remaining 15 grams per minute being recycled.

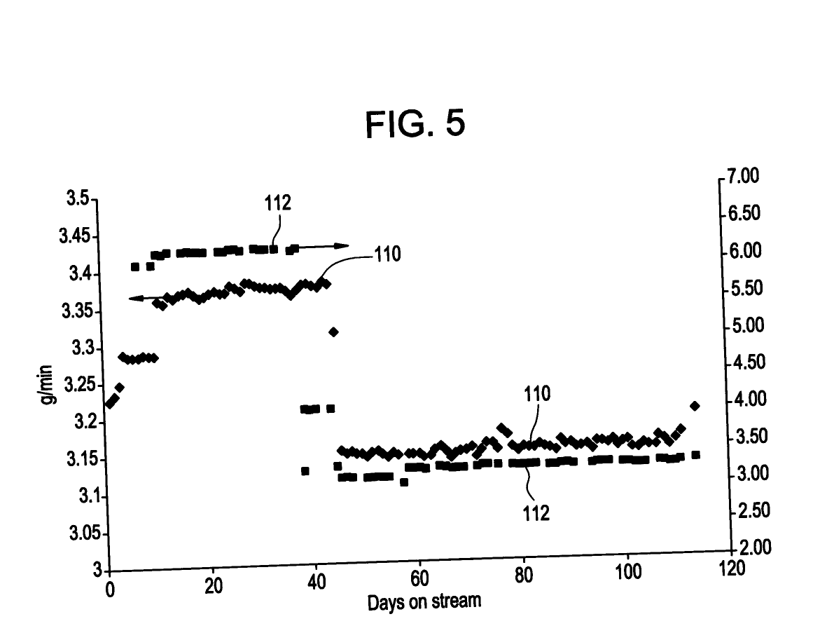

The results of this experimental work are illustrated in FIGS. 5-11. Turning initially to FIG. 5, curve 110 shows the benzene in grams per minute plotted on the ordinate versus the total cumulative days on stream plotted on the abscissa. Curve 112 is a corresponding plot for the benzene/ethylene mole ratio. As indicated in FIG. 5, at about 44 days the benzene rate was cut from a nominal value of about 3.35 to 3.4 grams per minute to a nominal value of about 3.15 grams per minute. The benzene ethylene mole ratio during this initial phase was about 5.7, and after the benzene rate was reduced the benzene ethylene mole ratio was reduced to a value of about 3.25.

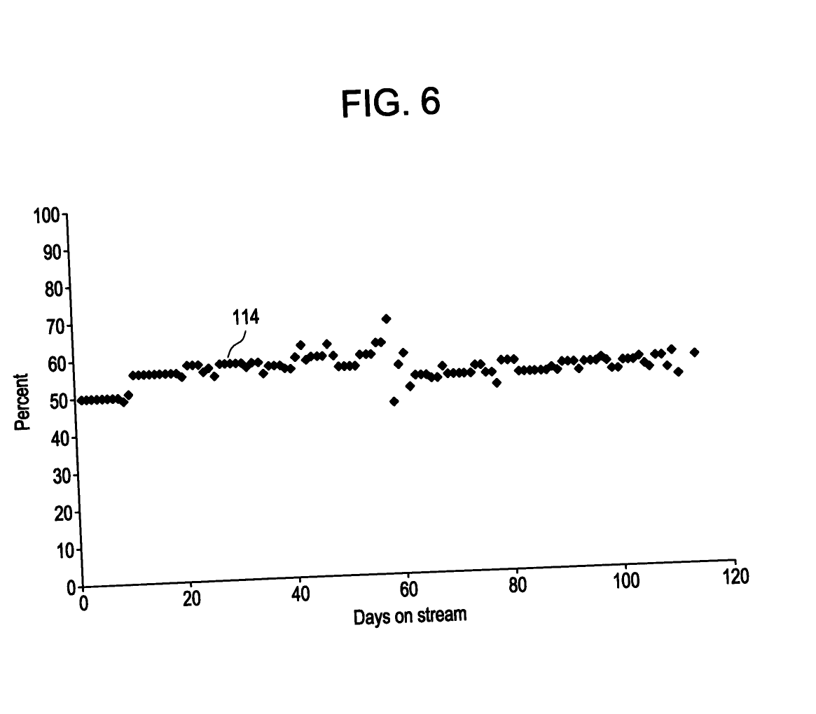

FIG. 6 shows the percent of the bed used in the catalytic reaction plotted on the ordinate versus the total days on stream plotted on the abscissa. The percent of the catalyst bed as indicated by curve 114 was calculated based upon the maximum temperature sensed across the bed employing six temperature sensors spaced from the inlet to the outlet of the reactor. As can be seen from an examination of FIG. 6, the cerium promoted zeolite beta catalyst was remarkably stable throughout the test run, and showed no need for regeneration.

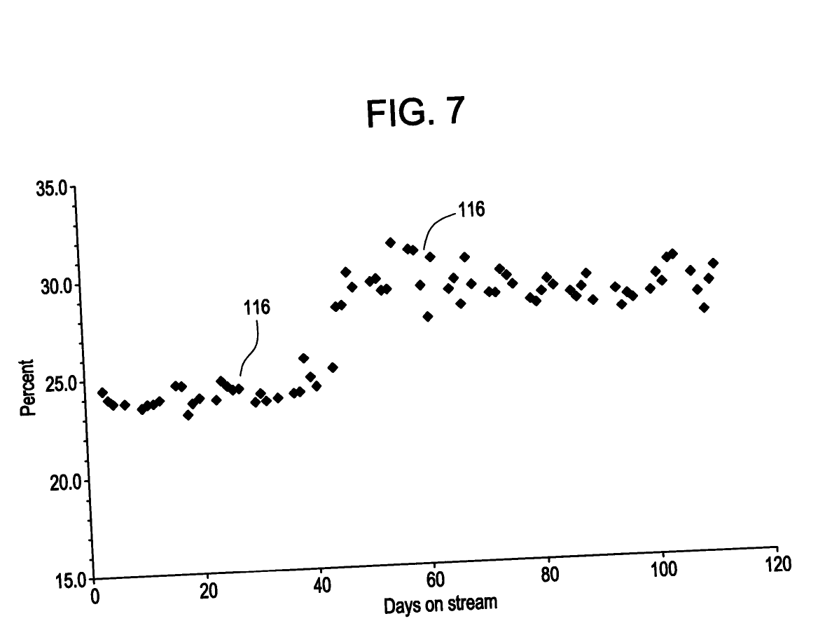

FIG. 7 illustrates the ethylbenzene equivalent yield in terms of percent conversion relative to benzene plotted on the ordinate versus the time of the run in days on the abscissa. As I indicated by curve 116, the ethylbenzene yield ranged from about 24-25%, and then increased to about 28-30% when the benzene yield was decreased to result in an increase in the benzene/ethylene mole ratio. In examining the data in FIG. 7, it should be recognized that the ethyl benzene yield is an equivalent yield relative to benzene, and not an absolute yield.

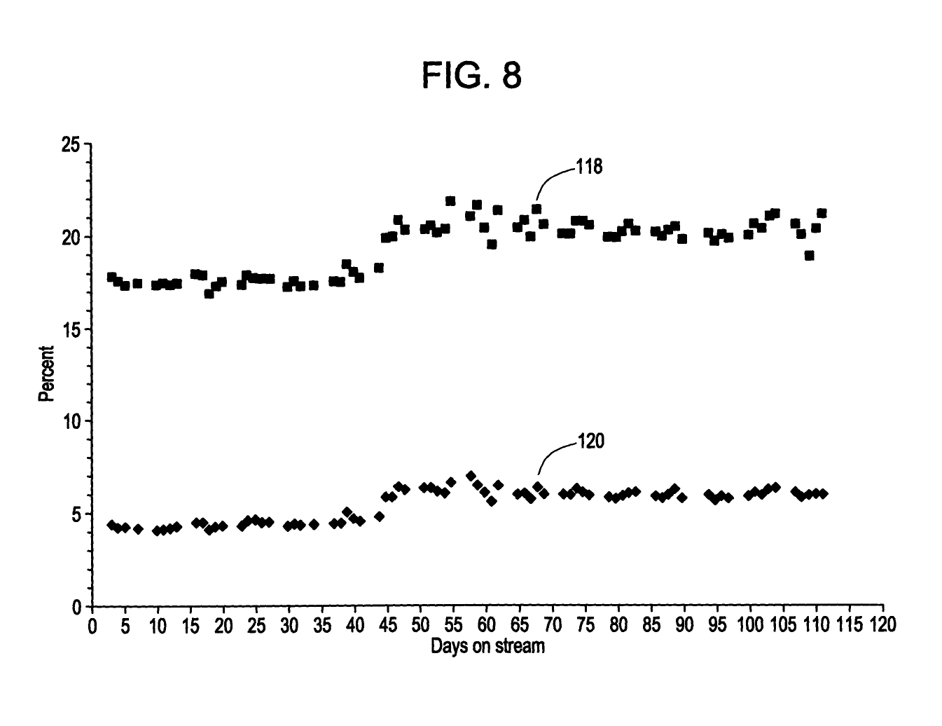

FIG. 8 shows the ethyl benzene yield and the diethyl benzene yield as a percentage of the total product output over the life of the reactor run. The ethylbenzene yield plotted as a percent of the product is indicated by curve 118 and the diethyl benzene yield plotted as a percent of a total product is indicated by curve 120. As indicated by curve 120, the diethyl benzene yield stayed relatively constant over the life of the run with only a proportionate increase corresponding to the ethylbenzene yield when the benzene/ethylene mole ratio was decreased at day 42.

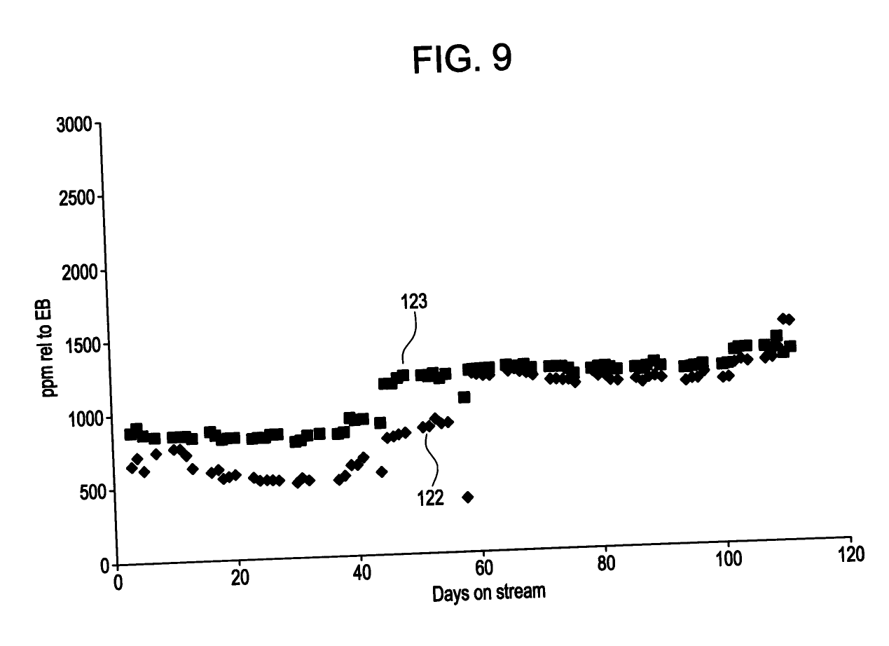

FIG. 9 shows the byproduct yield relative to ethylbenzene for propyl benzene indicated by curve 122, and butyl benzene indicated by curve 123. In FIG. 9, curves 122 and 123 are plots of the respective byproduct in terms of parts per million (ppm) relative to the ethylbenzene yield. As indicated by the data in FIG. 9, both propyl benzene and butyl benzene yields were less than 1,000 ppm during the initial portion of the yield and remained at values less than 1,500 ppm, in most cases about 1,200 ppm, after the benzene ethylene mole ratio was reduced.

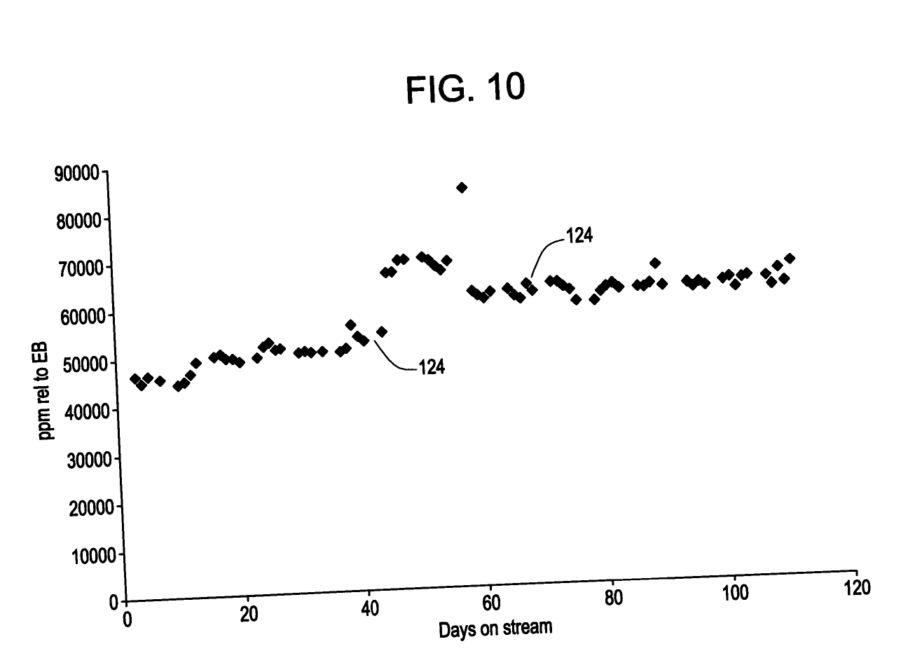

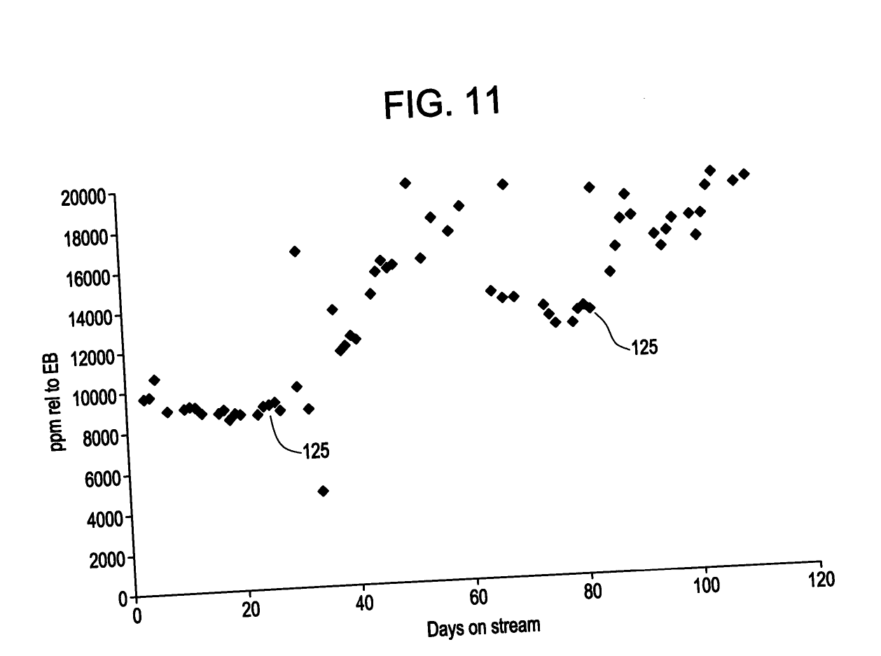

In FIG. 10, curve 124 shows the triethylbenzene yield in parts per million relative to ethylbenzene plotted on the ordinate versus the days of the run plotted on the abscissa. In FIG. 11, curve 125 shows the corresponding data for “heavies” (products having a molecular weight greater than triethylbenzene) in parts per million relative to ethylbenzene. While the data points in FIG. 11 are widely scattered, particularly after the decrease in the benzene/ethylene mole ratio, both the triethylbenzene and the “heavies” byproducts showed a response generally similar to the other byproduct yields. In all cases these yields for a given benzene ethylene mole ratio remained relatively constant and showed little or no progressive buildup which could be attributed to the recycle of the product from the alkylation reactor.

As noted previously, the recycle ratio for the experimental work as shown in FIGS. 5-11 was about 5:1. Operating at this relatively high ratio provided a solvent presence to solubalize the ethylene and a heat exchange presence to prevent the buildup of excessive heat within the reactor. At the same time this was accomplished without an excessive buildup of impurities notwithstanding, the relatively high recycle ratio of 5:1.

Having described specific embodiments of the present invention, it will be understood that modifications thereof may be suggested to those skilled in the art, and it is intended to cover all such modifications as fall within the scope of the appended claims.

What is claimed:

1. A method for the alkylation of an aromatic substrate comprising:
(a) providing an alkylation reaction zone containing a molecular sieve aromatic alkylation catalyst;
(b) introducing a feed stock comprising an aromatic substrate and an alkylating agent into the inlet of said alkylation reaction zone and into contact with said catalyst;
(c) operating said alkylation reaction zone at temperature and pressure conditions to cause alkylation of said aromatic substrate in the presence of said molecular sieve alkylation catalyst to produce an alkylation product comprising a mixture of said aromatic substrate and monoalkylated and polyalkylated aromatic components;
(d) withdrawing the alkylation product from said alkylation reaction zone;
(e) recycling a first portion of alkylation product withdrawn from said alkylation reaction zone back to said alkylation reaction zone and supplying said first portion to said reaction zone, along with said aromatic substrate and said alkylating agent; and
(f) supplying a second portion of said alkylation product to a recovery zone for the separation of a monoalkylated and polyalkylated aromatic components from said unreacted aromatic substrate.

2. The method of claim 1 wherein the weight ratio of said first portion to said second portion of said alkylation product is at least 1:1.

3. The method of claim 1 wherein the weight ratio of said first portion to said second portion of said alkylation product is at least 2:1.

4. The method of claim 1 wherein said alkylation reaction zone is operated at temperature and pressure conditions in which said aromatic substrate is in the liquid phase or in the supercritical phase.

5. The method of claim 4 wherein said alkylation reaction zone is operated under temperature and pressure conditions in which said aromatic substrate is in the supercritical phase.

6. The method of claim 5 wherein said aromatic substrate is benzene and said alkylating agent is ethylene and said molecular sieve aromatic alkylation catalyst comprises zeolite beta.

7. The method of claim 6 wherein said zeolite beta alkylation catalyst comprises a rare earth metal modified zeolite beta catalyst.

8. The process of claim 7 wherein said zeolite beta alkylation catalyst comprises a lanthanum modified zeolite beta.

9. The method of claim 7 wherein said zeolite beta alkylation catalyst comprises a cerium modified zeolite beta.

10. The method of claim 1 wherein at least a predominant portion of the alkylation catalyst in said alkylation reaction zone is contained within a single catalyst bed of said alkylation reaction zone.

11. The method of claim 10 wherein said alkylation reaction zone is operated under temperature and pressure conditions in which said aromatic substrate is in the supercritical phase.

12. The method of claim 11 wherein said aromatic substrate is benzene and said alkylating agent is ethylene and said molecular sieve aromatic alkylation catalyst comprises zeolite beta.

13. The method of claim 1 wherein said alkylation reaction zone comprises at least two spaced catalyst beds, each of said catalyst beds containing said molecular sieve aromatic alkylation catalysts.

14. The method of claim 13 wherein said first portion of said alkylation reaction product is divided into two subproducts, with the first of said subproducts recycled to the inlet of said alkylation reaction zone and into contact with a first of said catalyst beds, and a second of said subproducts is recycled to said alkylation reaction zone and introduced into said alkylation reaction zone between said first and second catalyst beds.

15. A method for the alkylation of benzene comprising
(a) providing an alkylation reaction zone containing a molecular sieve aromatic alkylation catalyst;
(b) supplying a feed stock comprising benzene and a C₂-C₄ alkylating agent to said alkylation reaction zone;
(c) operating said alkylation reaction zone at temperature and pressure conditions in which benzene is in the liquid phase or in the supercritical phase to cause alkylation of said benzene in the presence of said molecular sieve alkylation catalyst to produce an alkylation product comprising a mixture of benzene, monalkyl benzene and polyalkyl benzene;
(d) recovering the alkylation product from said alkylation reaction zone and supplying a first portion of said product to a recycle stream for introduction into said alkylation reaction zone and a second portion of said product to an intermediate recovery zone for the separation and recovery of alkyl benzene from the alkylation product and the separation and recovery of a polyalkylated aromatic component including a dialkylbenzene;
(e) supplying at least a portion of the polyalkylated aromatic component including said dialkyl benzene to a transalkylation reaction zone containing a molecular sieve transalkylation catalyst;
(f) supplying benzene to said transalkylation zone; and
(g) operating said transalkylation zone under temperature and pressure conditions to cause disproportionation of said polyalkylated aromatic to produce a disproportionation product having a reduced dialkyl benzene content and an enhanced alkyl benzene content.

16. The method of claim 15 wherein benzene is recovering from the alkylation product in said recovery zone and recycled to said alkylation reaction zone.

17. The method of claim 15 wherein said alkylation catalyst is a zeolite beta molecular sieve and said reaction zone is operated at temperature and pressure conditions in which benzene is in the supercritical phase.

18. The method of claim 17 wherein said zeolite beta alkylation catalyst is a zeolite beta modified by the inclusion of a lanthanide rare earth.

19. The method of claim 18 wherein said zeolite beta comprises a lanthanum-modified zeolite beta.

20. The method of claim 18 wherein said zeolite beta comprises a cerium-modified zeolite beta.

21. The method of claim 15 further comprising supplying at least a portion of said disproportionation product from said transalkylation zone to said intermediate recovery zone.

22. The method of claim 15 further comprising supplying at least a portion of said disproportionation product for recycle into said alkylation reaction zone.

23. The method of claim 22 wherein at least a portion of said recycled product from said transalkylation zone is supplied for recycle to said alkylation reaction zone and another portion of said disproportionation product is supplied to said intermediate recovery zone.
